(19) United States
(12) Patent Application Publication
(10) Pub. No.: US 2007/0066948 A1
(43) Pub. Date: Mar. 22, 2007

(54) Absorbent article with quilted feature

(75) Inventor: Carol L. Erdman, West Chester, PA (US)

Correspondence Address: GOSZ AND PARTNERS, LLP, 450 BEDFORD STREET, LEXINGTON, MA, 02420, US

(73) Assignee: Tyco Healthcare Retail Group, Inc.

(21) Appl. No.: 11/229,861

(22) Filed: Sep. 19, 2005

(51) Int. Cl. A61F 13/15

(52) U.S. Cl. 604/380; 604/367

(57) ABSTRACT

This disclosure provides an absorbent article having a high bulk material disposed between the body-facing surface and the garment facing surface of the article, wherein the high bulk material is relatively low cost and the article surface is embossed in the area of the high bulk material. Such an absorbent article desirably prevents leakage and/or has increased absorbency in the area of the high bulk material and is relatively easy and efficient to manufacture.

### BACKGROUND

1. Technical Field

The present disclosure generally relates to absorbent articles, and more particularly to an absorbent article having a high bulk, quilted feature that is aesthetically pleasing, while being inexpensive and easily manufactured.

2. Description of the Related Art

Absorbent articles such as, for example, disposable diapers, adult incontinent pads, sanitary napkins, pantiliners, incontinent garments and the like are generally worn, in cooperation with garments and disposed against a body surface by infants or adult incontinent individuals. The absorbent article is employed to collect and absorb body fluid discharge, such as, for example, blood, menses, urine, aqueous body fluids, mucus, cellular debris and other forms of body discharge. For example, the absorbent article may be disposed between the legs of an individual adjacent a crotch area. The absorbent article is positioned with a garment and drawn into engagement with a body surface of the crotch area to collect body discharge.

As is known, absorbent articles typically include a fluid permeable coverstock for engaging the body surface, a fluid impermeable backsheet and an absorbent core supported therebetween. The backsheet serves as a moisture barrier to prevent fluid leakage to the garment. The absorbent core usually includes a liquid retention material that faces the body surface. The absorbent core can include, for example, loosely formed cellulosic fibers, such as, for example, wood pulp, rayon, or cotton for acquiring and storing body discharge. The absorbent core may also include tissue wrapping or wadding, and/or super absorbent polymer.

Some absorbent article designs typically incorporate elastic elements in the waist, stomach, and leg areas for improving the fit, aesthetics and leakage resistance. The waist and stomach elastic elements increase the flexibility of the absorbent article, allowing the same article to accommodate a greater range of body sizes. In addition, they make the article more form-fitting for the wearer. The topsheet and backsheet are joined to support the components of the absorbent article.

Infants, toddlers, and other incontinent individuals wear disposable absorbent articles such as diapers and training pants, which include “pull-on” or “pant-type” garments having fixed sides, single-use diaper garments including front and back portions joined, usually by an adhesive, when the diaper is initially donned, and hybrid garments which are donned like a diaper garment but are designed to be removed and replaced like a pant-type garment.

It would therefore be desirable to provide an absorbent article having greater absorbent properties, reduces leaks, is aesthetically pleasing to the wearer, and/or is inexpensive to manufacture.

### SUMMARY

Accordingly, an absorbent article is provided having a high bulk material disposed between the body-facing surface and the garment facing surface of the article. The high bulk material is relatively low cost. The absorbent article surface may be embossed in the area of the high bulk material to provide a cushioned, quilted appearance. Such an absorbent article desirably prevents leakage and/or has increased absorbency in the area of the high bulk material and is relatively easy and efficient to manufacture. Objects and advantages of the present disclosure are set forth in part herein and in part will be obvious therefrom, or may be learned by practice of the present disclosure that is realized and attained by the instrumentalities and combinations pointed out in the appended claims for the devices and methods of the present disclosure consisting of its constituent parts, constructions, arrangements, combinations, steps and improvements herein shown and described.

In one particular embodiment, an absorbent article is provided that comprises a body-facing surface, a garment-facing surface, and an absorbent core, wherein the absorbent core is disposed between the body-facing surface and the garment-facing surface. A high bulk material is disposed between the body-facing surface and the garment-facing surface. The garment-facing surface and/or the body-facing surface overlay the high bulk material and may be embossed. Embossing is preferably in a quilted pattern.

The high bulk material may include an absorbent material or a hydrophobic (non-absorbent) material. In either case, the material is preferably a non-woven material. In other preferred embodiments, the high bulk material is creped. Absorbent high bulk materials suitable for use according to the principles of this disclosure include, for example, wood pulp, cellulose wadding, cotton fibers which are, preferably, pressed into creped sheets. Hydrophobic high bulk materials suitable for use according to the principles of this disclosure include, for example, polyethylenes, polypropylenes, polystyrenes, and polyurethanes.

In one embodiment, the high bulk material is disposed between an absorbent core and a fluid-impermeable backsheet (e.g., the garment facing surface).

In another embodiment, the absorbent high bulk material is disposed between an absorbent core and a fluid-permeable portion of the body-facing surface (i.e., a topsheet). Preferably, the high bulk material has a higher absorbent rate than the absorbent core and acts as an acquisition layer.

In another preferred embodiment, a creped high bulk material (absorbent or hydrophobic) is disposed adjacent to a leg gather, or in the waistband region of the absorbent article. In another embodiment, the leg gather is formed from a creped high bulk material (absorbent or hydrophobic).

### BRIEF DESCRIPTION OF THE DRAWINGS

The objects and features of the present disclosure are set forth with particularity in the appended claims. The present disclosure, as to its organization and manner of operation, together with further objectives and advantages may be understood by reference to the following description, taken in connection with the accompanying drawings, in which:

FIG. 1 is a schematic cross section of an absorbent article in accordance with the principles of this disclosure;

FIG. 2 is a schematic cross section of an absorbent article in accordance with the principles of this disclosure; and

FIG. 3 is a plan view of an absorbent article having embossing in a quilted pattern.

### DETAILED DESCRIPTION OF THE EXEMPLARY EMBODIMENTS

The exemplary embodiments of the absorbent article are discussed in terms of fluid absorbent articles, and more particularly, in terms of an absorbent article having a high bulk material disposed between the body-facing surface and the garment-facing surface. The presently disclosed absorbent article is contemplated to be employed with, for example, training pants, adult incontinent products, disposable diapers, and other incontinent articles.

In the discussion that follows, the term “body-facing surface” refers to a portion of a structure that is oriented towards a body surface, and the “garment-facing surface” refers to a portion of the structure that is oriented towards a garment and is typically opposing the body-facing surface and may be referred to as such. As used herein, the term “body surface” refers to a portion of an individual's body that the absorbent article is disposed with for collecting and absorbing fluid discharge from the individual. As used herein, the term “absorbent article,” or “absorbent garment” refers to garments that absorb and contain body waste, and more specifically, refers to garments that are placed against or in proximity to the body of the wearer to absorb and contain the various body discharges and wastes.

A non-exhaustive list of examples of absorbent articles includes training pants, diapers, diaper covers, disposable diapers, feminine hygiene products and adult incontinence products. The term absorbent articles includes all variations of absorbent garments, including disposable absorbent garments that are intended to be discarded or partially discarded after a single use and unitary disposable absorbent garments that have essentially a single structure. As used herein, the term “training pants” refers to an absorbent article generally worn by children about the lower torso. The absorbent article may be disposable or partially discarded after a single use. The absorbent article can be a unitary structure or include replaceable inserts or interchangeable parts.

Absorbent articles and diapers may have a number of different constructions. In each of these constructions it is generally the case that an absorbent core is disposed between a liquid pervious, body-facing top sheet which forms at least a portion of the body-facing surface, and a liquid impervious, exterior facing back sheet. In some cases, one or both of the top sheet and back sheet may be shaped to form a pant-like article, such as a training pant. In other cases, the top sheet, back sheet and absorbent core may be formed as a discrete assembly that is placed on a main chassis layer and the chassis layer is shaped to form a pant-like absorbent article. The absorbent article may be provided to the consumer in the fully assembled pant-like shape or may be partially pant-like and require the consumer to take the final steps necessary to form the final pant-like shape. Training pants are typically fully formed and applied by pulling the absorbent article over a child's legs. For a diaper application, an open article is provided and wrapped about a child's waist, such as by manually fastening one or more fasteners or fastener tabs.

The following discussion includes a description of the disposable absorbent article in accordance with the present disclosure.

In a first embodiment, the high bulk material is placed in the waistband region of the absorbent article to improve the functionality and/or the aesthetics of the absorbent article. The waistband region is characterized by an opening for the wearer's torso and contains one or more elastic members or equivalent structure in order to secure the absorbent article about the wearer. Some absorbent articles also contain waistband foam in order to provide padding for the wearer by disbursing the elastic forces across a wider area, as well as providing aesthetic appeal.

The high bulk material is inserted between the body-facing surface and the garment-facing surface and then an area on both of the body-facing and garment facing surfaces is embossed through all layers of the absorbent article, preferably in a quilted pattern. It is contemplated that only one or a plurality of layers of the absorbent article may be embossed. It is further contemplated that only the body facing surface and/or the garment facing surface may be embossed. In one embodiment, the high bulk material is inserted between the absorbent core and the garment-facing surface. Alternatively, the high bulk material is inserted between the absorbent core and the body-facing surface. The high bulk material may be used in addition to, or instead of the waistband foam. In this configuration the high bulk material provides an additional leak guard function. High bulk materials that are absorbent act as a leak guard by providing absorbent capacity in excess of that available from the absorbent cores. This is particularly useful in instances when the void volume is unexpectedly high and the absorbent cores become saturated. High bulk materials that are hydrophobic also act as a leak guard by repelling excess fluid away from the waistband region and back toward the absorbent cores. This function is particularly useful under conditions where the wearer's void rate is greater than the rate of absorption of the absorbent cores. The waistband hydrophobic high bulk material helps to contain the excess volume between the body-facing surface and the garment-facing surface for a time sufficient for the absorbent cores to trap the entire voided volume. In another embodiment, the high bulk material may form the inner leg gathers. The high bulk material advantageously provides improved conformance to the legs of the wearer, and improves containment of leakage through the inner leg gather.

In one embodiment, the high bulk material is in the form of non-woven sheets. Preferably, the high bulk material sheets are creped to add bulk to the material without requiring additional material. The high bulk material sheets may be creped (crinkled) during the manufacture of the material, or the sheets may be creped by repeated folding in a Z-folded pattern.

In another embodiment, the region or area of the absorbent article that supports the high bulk material and the portions of the layers of the absorbent article disposed adjacent thereto are embossed in an aesthetically pleasing pattern, such as a quilted pattern. Similar to that discussed, the area on both the body-facing and garment facing surfaces is embossed through all layers of the absorbent article. Embossing may be done by, for example, thermal tacking and ultrasonic tacking. It is envisioned that embossing may be performed on the surface (i.e., garment-facing surface or body facing surface) of the absorbent article that is closest to the high bulk material. The embossing advantageously enhances the aesthetic quality of the embossed surface, holds the high bulk material in place, and enhances the “cushy” feel imparted by the high bulk material.

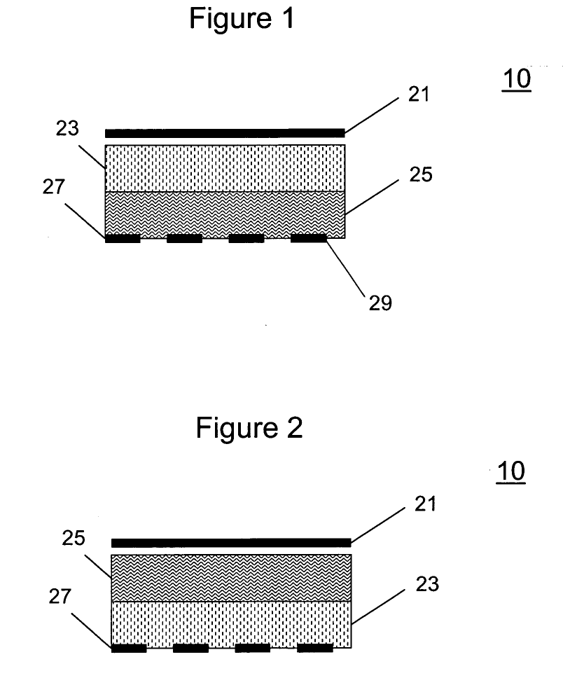

FIG. 1 is a schematic cross-section of an absorbent article 10 in which the absorbent high bulk material 25 is disposed between the absorbent core(s) 23 and the body-facing surface 27, preferably in a region where the body-facing surface 27 consists of a fluid-permeable topsheet 29. Desirably, the high bulk material 25 has a higher rate of absorption, but a lower fluid capacity compared to the absorbent core 23. In this manner, the high bulk material acts as an acquisition layer to quickly absorb any fluid to prevent leakage, and facilitate the fluid transfer into the high capacity absorbent core(s) 23.

FIG. 2 is a schematic cross-section of an absorbent article 10 in which the absorbent high bulk material 25 is disposed between the absorbent core(s) 23 and the garment-facing surface 21. In this configuration, the high bulk material 25 provides a “cushy” appearance, imparting enhanced aesthetic qualities to the article. The absorbent high bulk material 25 also increases the absorbent capacity of the article 10.

In another embodiment, creped high bulk material is disposed around a leg opening of the absorbent article. Leg openings are characterized by elastic members or equivalents which gather the material of the absorbent article against the skin of the wearer and prevent leaks. Disposing the creped high bulk material around the leg opening in a manner such that the creping causes the high bulk material to constrict around the wearer's leg. This property of creped materials helps secure the leg openings about the wearer and prevent leaks without requiring additional elastic members. The creped high bulk material may be absorbent or hydrophobic according to the principles described above for the waistband high bulk material configuration.

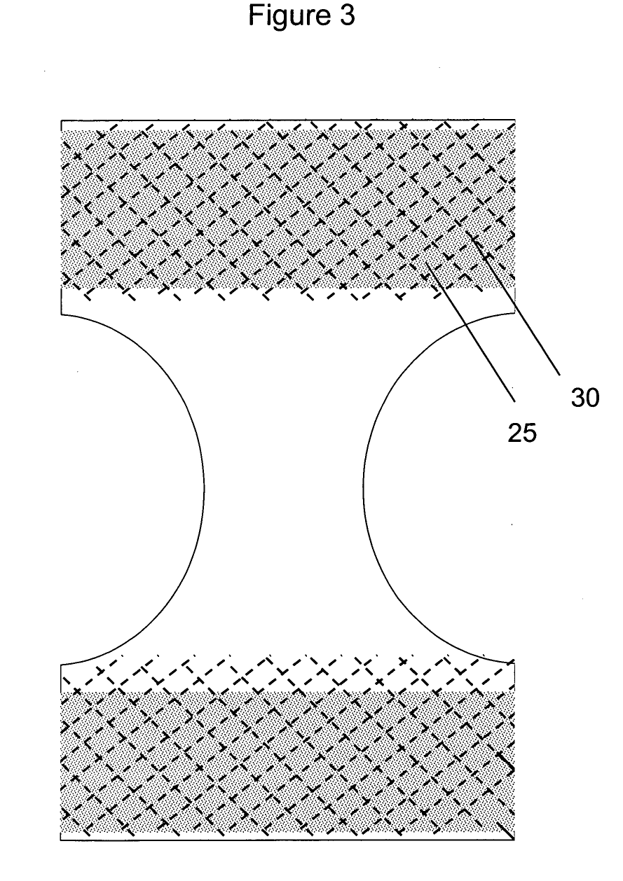

FIG. 3 is a plan view of an absorbent article having an area on both the body facing and garment-facing surface embossed through all layers of the absorbent article, as described. The embossing 30 is in a quilted pattern in the area of the absorbent article that is overlying the high bulk material 25.

For all of the foregoing embodiments, it is preferred that the area on both the body-facing surface and the garment facing surface that overlies the high bulk material is embossed through all layers of the absorbent article. The embossing may be done by any method known in the art including, for example, ultrasonic bonding or thermal bonding.

Fluid permeable top sheets suitable for use with disposable absorbent articles similar to those of the present disclosure are well known in the art. The top sheet is sufficiently pervious to permit liquids (e.g., urine) to readily penetrate. Suitable top sheets may be woven or nonwoven materials, polymeric materials (e.g., apertured formed thermoplastic films, apertured plastic films, and hydroformed thermoplastic films), porous foams, reticulated foams, reticulated thermoplastic films, and thermoplastic scrims. Suitable woven and nonwoven materials include natural fibers (e.g., wood and cotton fibers), synthetic fibers (e.g., polymeric fibers such as polyester, polypropylene, and polyethylene fibers). In other embodiments, the top sheet may be treated with a surfactant to facilitate the transfer of fluid through the top sheet into the absorbent core.

Absorbent core materials and configurations for use with disposable absorbent articles similar to those of the present disclosure are also well known in the art. The absorbent core may be configured as a single continuous core or several discontinuous cores that may or may not be connected in fluid contact. The absorbent core may be symmetrical or asymmetrical and, specifically, may be configured to be gender specific. The shape of the absorbent core will depend upon the specific material used, the specific application for which the article is used, and the desired performance characteristics. The absorbent core(s) may consist of either a single type of absorbent material or may contain multiple types of absorbent materials. Suitable materials for the absorbent core include, for example, wood pulp, cellulose wadding, cotton fibers, absorbent foams, sponges, superabsorbent polymers (e.g., hydrogels), absorbent gelling materials, or combinations thereof.

Fluid impermeable back sheets for use with disposable absorbent articles similar to those of the present disclosure are also well known in the art. Preferably, the back sheet is a relatively thin (e.g., about 0.012 mm to about 0.051) plastic film. It is particularly desirable to use back sheets which, while being impervious to aqueous fluids, allows vapors to escape. Suitable material for such back sheets include, for example, microporous polyethylene and polypropylene films. In other configurations, the back sheet may be a laminate consisting of a fluid impervious film and a woven or nonwoven outer cover. There is no requirement that the outer cover be fluid impervious. Frequently the outer cover is selected for its aesthetic properties.

The disposable absorbent article of this disclosure may include structure to fasten the front and back side panels around the torso of the wearer. Such structure may include, for example, hooks, snap fasteners, and unilateral or bilateral external adhesive tabs. Alternatively, an adhesive may be applied directly to the inside face (e.g., directly on the body facing top sheet or by way of an adhesive-containing strip affixed to the top sheet) of either the front or back side panel. The adhesive is exposed by removing a protective film covering and the inner face of the adhesive-containing side panel is adhered to the outer face (back sheet) of the opposite side panel to form a side seam.

The assembly of the absorbent article may be performed according to well known principles in the art and may be varied depending upon the desired performance characteristics. For example, the absorbent cores may be “free floating” between the top sheet and back sheet, it may be adhered to the back sheet, or it may be elastically supported. The top sheet and back sheet may be joined by, for example, an adhesive or heat sealing (melting) the materials together. This process encapsulates the absorbent cores and all of the elastic members within the absorbent article to provide a unitary structure that is aesthetically appealing.

Although the foregoing disclosure has been described in some detail by way of illustration and example for purposes of clarity of understanding, it will be readily apparent to those of ordinary skill in the art in light of the teachings of this present disclosure that certain changes and modifications may be made thereto without departing from the spirit or scope of the appended claims.

What is claimed is:

1. An absorbent article comprising:
(i) a plurality of layers including a body-facing surface, a garment-facing surface, and an absorbent core, wherein said absorbent core is disposed between said body-facing surface and said garment-facing surface; and
(ii) a high bulk material disposed between said body-facing surface and said garment-facing surface, wherein an area on the surfaces adjacent said high bulk material is embossed through the layers of the absorbent article.

2. The absorbent article of claim 1, wherein said high bulk material is an absorbent material.

3. The absorbent article of claim 1, wherein said high bulk material is a nonwoven material.

4. The absorbent article of claim 1, wherein said high bulk material includes a creped material.

5. The absorbent article of claim 4, further comprising leg gathers that include said creped material.

6. The absorbent article of claim 1, wherein said high bulk material is disposed between said absorbent core and said body-facing surface.

7. The absorbent article of claim 4, wherein said high bulk material is disposed adjacent to a leg gather.

8. The absorbent article of claim 1, wherein said high bulk material is present in the waistband area of said absorbent article.

9. The absorbent article of claim 8, wherein said high bulk material is hydrophobic.

10. An absorbent article comprising:
(i) a plurality of layers including a body-facing surface, a garment-facing surface, and an absorbent core, wherein said absorbent core is disposed between said body-facing surface and said garment-facing surface; and
(ii) a creped high bulk material disposed between said body-facing surface and said garment-facing surface, wherein an area on the surface adjacent said high bulk material is embossed through the layers of the absorbent article.

11. The absorbent article of claim 10, wherein said creped high bulk material is in the waistband area or adjacent to a leg gather.

12. The absorbent article of claim 10, wherein said creped high bulk material is disposed between said absorbent core and said body-facing surface.

13. The absorbent article of claim 10, wherein said creped high bulk material is disposed in the waistband region.

14. The absorbent article of claim 13, wherein said creped high bulk material is hydrophobic.

15. The absorbent article of claim 10, further comprising leg gathers including said creped high bulk material.

16. The absorbent article of claim 10, wherein said creped high bulk material has a repeating Z-fold configuration.
